(19) United States
(12) Patent Application Publication
(10) Pub. No.: US 2009/0306506 A1
(43) Pub. Date: Dec. 10, 2009

(54) Method Of Aquiring Data In The Mouth Of A Patient, Such A Device, An Arrangement Comprising A Dentist's Chair And Such A Device And The Use Of This Device

(75) Inventors: Stefan Heger, Aachen (DE); Peter Latzke, Aachen (DE); Klaus Radermacher, Stolberg (DE); Hubertus Spiekermann, Haan (DE); Joachim Tinschert, Aachen (DE)

Correspondence Address: K.F. ROSS P.C., 5683 RIVERDALE AVENUE, SUITE 203 BOX 900, BRONX, NY, 10471-0900, US

(73) Assignee: RHEINISCH-WESTFAELISCH-TECHNISCHE HOCHSCHULE AACHEN, Aachen (DE)

(21) Appl. No.: 12/301,622

(22) PCT Filed: May 30, 2007
(86) PCT No.: PCT/EP2007/004756

§ 371 Date: Apr. 25, 2009

(30) Foreign Application Priority Data
May 31, 2006 (DE) 10 2006 025 775.8

(51) Int. Cl. A61B 8/14

(52) U.S. Cl. 600/443

(57) ABSTRACT

A device (10) for acquiring data in the mouth of a patient, wherein the device (10) comprises an ultrasound sensor (20) and a support structure. The ultrasound sensor (20) is stored by means of the support structure when not in use and contains ultrasound deflection means (12) which are movable. A coupling body (21) is provided, which is arranged between the ultrasound deflection means (12) and a tooth area (1, 2, 3) or remaining tooth area to be swept over Excitation signals (14) are sent to the ultrasound sensor (20) and the ultrasound deflection means (12) are moved in order to thus produce an ultrasonic wave which sweeps over at least part of the tooth area (1, 2, 3) or remaining tooth area.

The invention relates to a method of acquiring data in the mouth of a patient by means of an ultrasound sensor according to the preamble of claim 1 and corresponding apparatuses, systems and the use thereof.

Nowadays the mold of a tooth, tooth area or residual tooth area is often taken with special elastomer impression materials directly in the mouth of the patient at the dentist, oral surgeon or orthodontist. These materials are applied, for example, in a tray that is then pressed on the tooth area of which an impression is to be made in the patient's mouth. The tray must be kept still while the material hardens. If this is not done, errors can occur. After hardening, the impression thus obtained can be removed from the mouth and processed further. Typically, a plaster model is produced in a further step. Errors can occur during this step, too.

The process described is time-consuming and often very unpleasant for the patient. Moreover, the cost of the material and the time necessary are relatively high and the results of the impression are sometimes inexact. Furthermore, the application of this approach in the patient's mouth can lead to gingival recessions and the loss of fibrous attachment. With the described conventional impression technique, problems can also occur through a contamination by blood, saliva and sulcus fluid of the surface from which the impression is to be made.

However, a CCD-based system for so-called intraoral data acquisition already exists today that at least in some cases can be used to replace the impression process described above. Details can be taken from U.S. Pat. No. 6,169,781 and the corresponding German utility model DE 29717432. However, with this system only one single prepared tooth can be recorded intraorally, i.e. in the patient's mouth, which tooth has been prepared for an inlay, onlay or an individual crown. However, time-intensive preparations are necessary when using this system, since blood as well as saliva or gums can distort the data acquisition. The tooth to be recorded must be completely dried by means of a cofferdam and under some circumstances the preparation border must be exposed, which can be painful for the patient. In addition, the corresponding tooth should be treated with a special powder in order to avoid reflections during data acquisition. This system thus entails several disadvantages that on the one hand are reflected in relatively high costs and that on the other hand also lead to pain or at least discomfort for the patient.

Another system is known from the German utility model DE 9319391 that records data by means of a CCD image sensor in the mouth of a patient. The image sensor shown is relatively large.

Another disadvantage of the CCD-based systems referenced is that only individual images can always be recorded, i.e. the geometry of only one single tooth can be determined. Thus only minor tooth restorations, e.g. inlays or individual crowns, can be made using this system. Another restriction of the system described is that the margins of the tooth to be recorded must lie supragingivally.

Moreover, there are imaging systems that work by means of x-rays. Through these systems an exposure of the patient to x-rays can occur which is considered to be not unproblematic.

There is already a variety of ultrasound systems that are used in the medical field. However, typical disadvantages are that no images are possible behind bones (because of the total reflection). Moreover, the behavior of the ultrasound wave is very complex.

In the dental field ultrasound methods have hitherto been used experimentally for sonoerosive processing of advanced technical ceramics, for densimetry and characterization of dental substances, for the representation of periodontal structures or for removing tartar and plaque. One example of a system for determining periodontal pockets in the area between teeth and gums can be derived from U.S. Pat. No. 5,100,318. The ultrasonic head used has an aperture in the millimeter range and must be placed directly on the gingiva in order to be able to detect a gum pocket. Another very similar system can be taken from U.S. Pat. No. 5,755,571.

Another system that is proposed for detecting individual teeth or dental arches is disclosed by U.S. Pat. Nos. 6,050,821 and 6,638,219. According to these US patents, a lining up of several ultrasonic transducers is used which perform a movement jointly during scanning in order to be able to record the teeth from several sides. The ultrasonic transducers sit in a type of cap or in a shell that is placed over the teeth to be examined. It is proposed to rinse the ultrasonic transducer with water in order to thus provide a coupling. However, since the cap or shell cannot be completely sealed with respect to the teeth or the dental lamina, water must be constantly added.

Overall, this approach is also laborious and imprecise. Furthermore, the entire system is expensive and complex. A reproducibility of measurements once taken and a comparability of data recorded are hardly possible.

Various ultrasound-based devices are known from published PCT patent application WO 2005/034785 [U.S. Pat. No. 7,285,093] in order to be able to scan the jaw or the inner structure of a jaw area. One of these devices is particularly suitable, for example, in order to be able to image the course of a nerve fiber in the jaw. The proposed structure is relatively complicated and a position sensor must be used in addition to the actual ultrasonic sensor system. The achievable recording area is relatively small.

One object of the invention is thus to provide a method and a corresponding apparatus that avoid or at least reduce the disadvantages of the approaches previously known. Furthermore, the system should be simple and uncomplicated in order to thus render possible a use by less well-trained operators. Moreover, the object of the invention is to provide a cost-effective apparatus. However, above all the focus is on ensuring that the application of the method and the use of the apparatus is not unpleasant for the patient and as far as possible does not have any side effects. Moreover, reproducibility of data acquisitions is important.

It is also important that the invention can easily be used for detection in the area of the front teeth as well as in the rear molar area.

This object is attained according to the invention through the features of the characterizing portion of method claim 1. Advantageous further developments and properties of the method according to the invention are defined by claims 2 through 24.

This object is also attained through the features of the characterizing portion of apparatus claim 25. Advantageous further developments and properties of the apparatus according to the invention are defined by patent claims 26 through 40.

This object is also attained through the features of the characterizing portion of apparatus claim 41 directed at a system and of use claims 42 and 43.

Details and advantages of the invention are described extensively below based on the specification and with reference to the drawing. Therein:

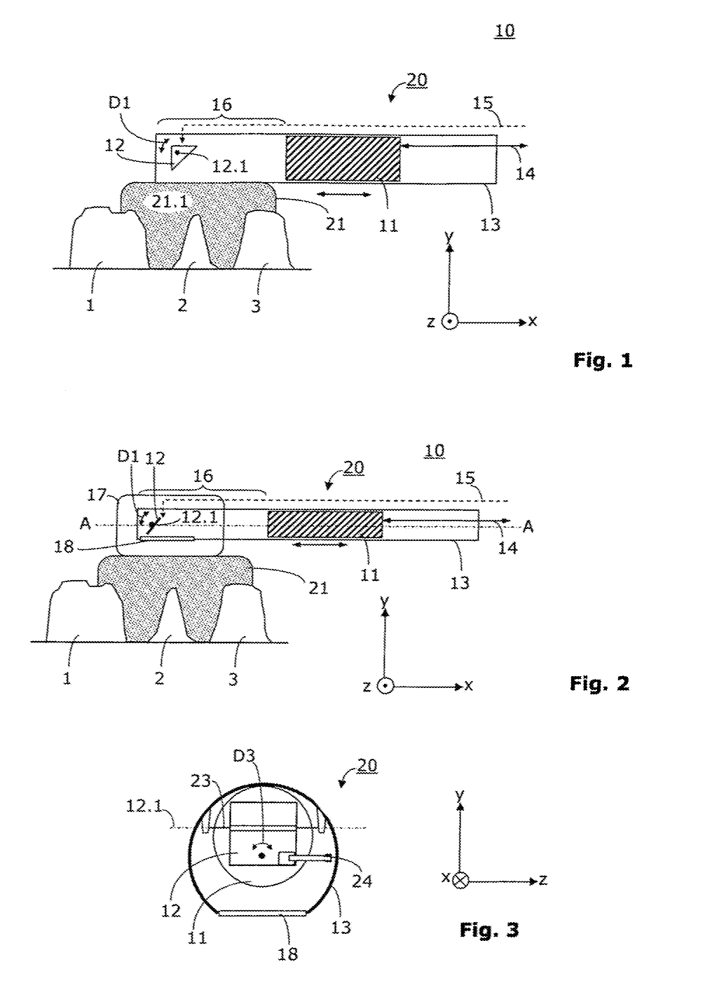

FIG. 1 is a diagrammatic side view of a tooth area and sectional view of an apparatus according to the invention;

FIG. 2 is a diagrammatic side view of a tooth area and sectional view of another apparatus according to the invention;

FIG. 3 is a diagrammatic side view of another apparatus according to the invention;

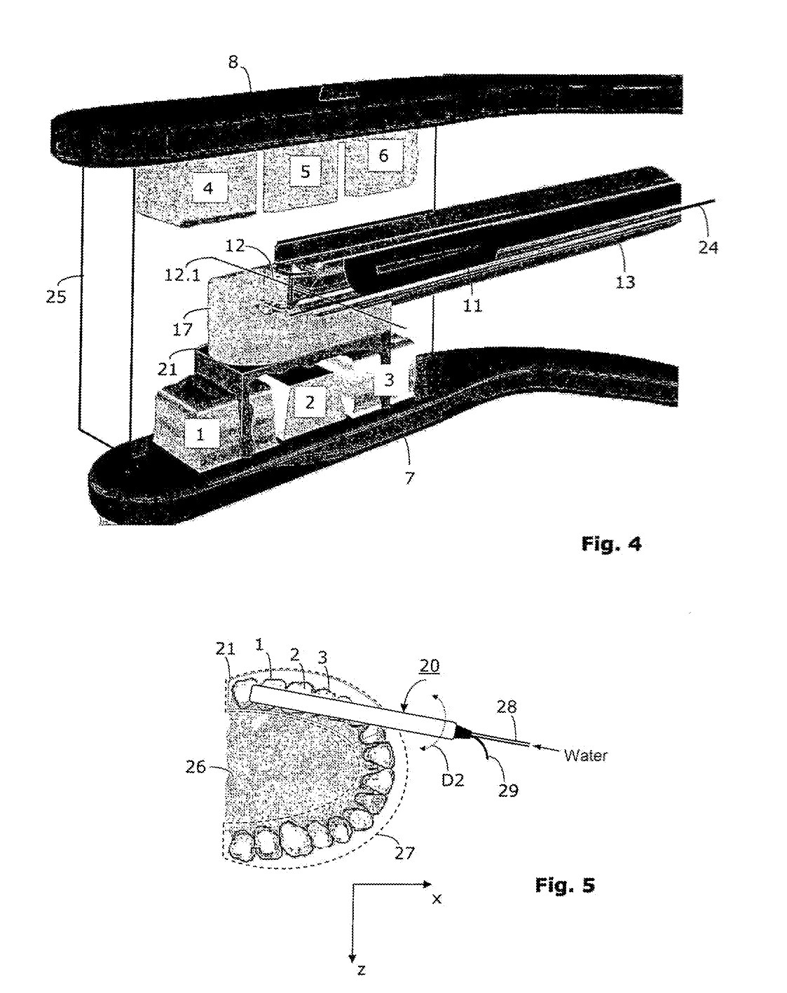

FIG. 4 is a diagrammatic perspective view of an upper jaw and a lower jaw with a tooth area and a sectional view of another apparatus according to the invention;

FIG. 5 is a diagrammatic plan view of a lower jaw with another apparatus according to the invention;

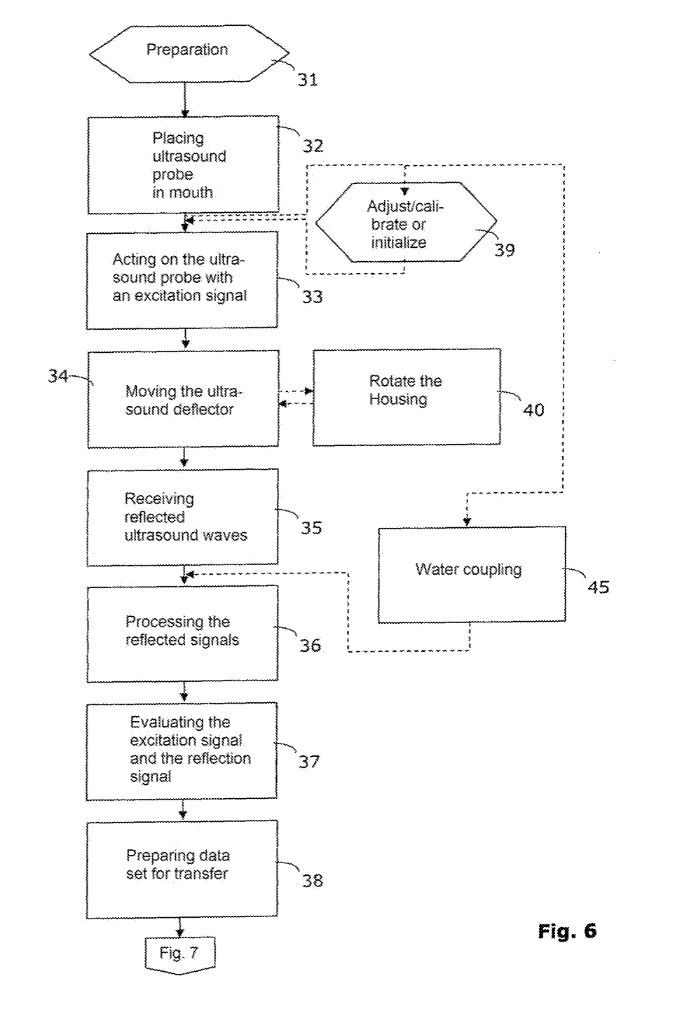

FIG. 6 is a flow chart of a first method according to the invention;

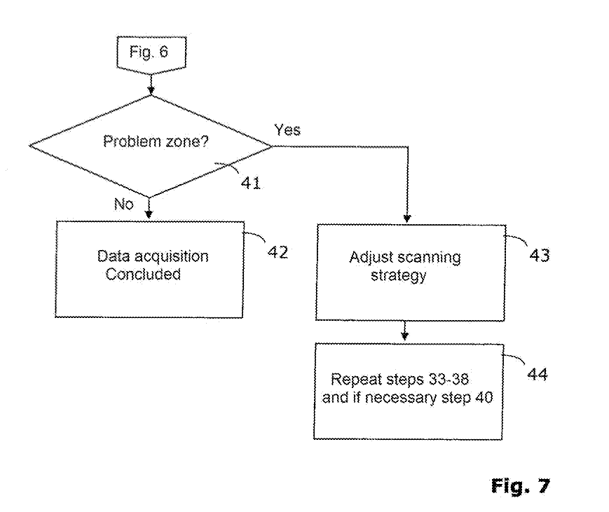

FIG. 7 is a flow chart of an extension of/addition to the first method according to the invention;

FIG. 8 is a flow chart of another method according to the invention;

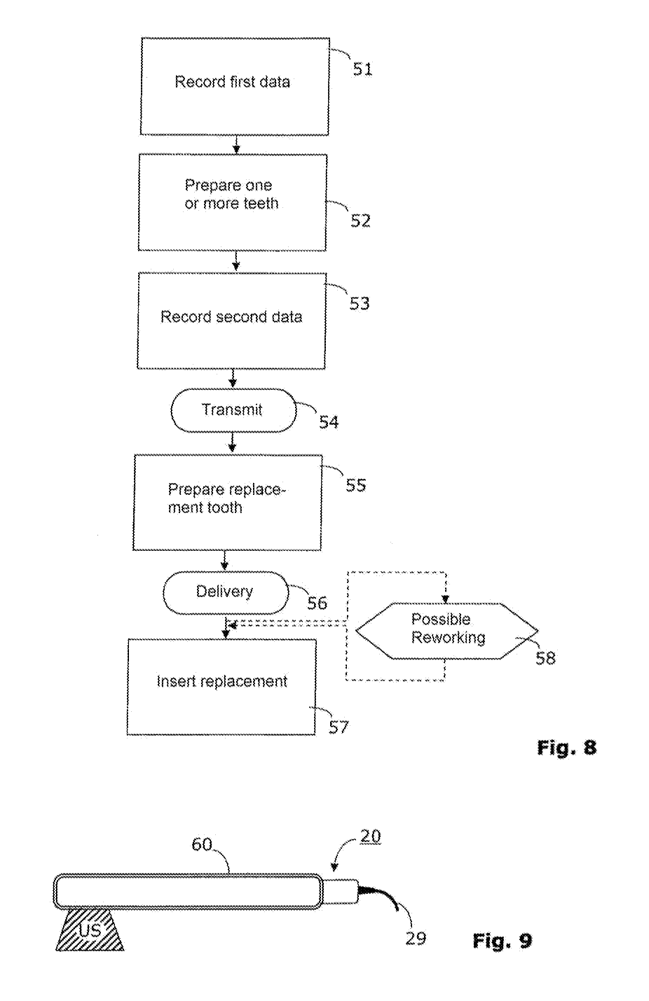

FIG. 9 is a diagrammatic side view of another embodiment of the invention.

The terms tooth area and residual tooth area are used below. These terms are to be understood to be synonymous with the following areas in a patient's mouth:
an individual tooth,
a dental arch with more than one individual tooth,
a tooth that is damaged or has been processed (e.g. a tooth with a cavity or a ground stump of a tooth),
a preparation for placing or fixing a crown or bridge (for example, a jaw implant)
a gap between the teeth,
a section of the jaw (with or without teeth),
a section of a jaw bone,
removable or fixed dentures,
an implant (for example a subperiosteal implant),
a supraconstruction for placing on an implant,
a dental brace (bracket).

In connection with the present invention a so-called ultrasound sensor 20 is mentioned on various occasions that comprises a probe 11. Examples are given below for ultrasound sensors that can be used in apparatuses 10 and systems according to the invention.

The term ultrasound in general covers the range between 16 kHz-1 GHz, where work is preferably carried out in the range between 1 MHz and 80 MHz in connection with the invention. The range between 40 MHz and 50 MHz average frequency is particularly preferred with ultrasonic transducers that operate in the impulse echo method, since a good resolution can be obtained in this range. In general the required ultrasonic wave can be generated piezoelectrically except with the electromagnetic ultrasonic transducers mentioned later.

Typically a distinction is made between ultrasound sensors that emit longitudinal waves and vertically polarized transverse waves (Sv waves) and those ultrasound sensors that emit horizontally polarized transverse waves (SH waves).

These longitudinal waves can be generated with liquid or gel-like coupling means between the probe 11 (for example, in the form of a piezoelectric probe) and the material to be scanned.

The horizontally polarized transverse waves (SH waves) were previously used more rarely, since they were generated only by conventional probes that required very viscous or solid coupling means, which rendered testing head movements impossible for most applications. Special coupling means are therefore required when working with transverse waves, since it is virtually impossible for transverse waves to expand in liquids (for example, water).

The apparatus according to the invention is characterized in that the actual probe 11 is essentially at rest during the measurement, i.e. the probe 11 does not move while the measurement is being carried out. Depending on the embodiment, however, during initialization (for example, for adjusting/repositioning the scanning area or the focusing), the probe 11 can be moved in a straight line in the direction of ultrasound deflection means 12 or away therefrom. After this initialization movement, the probe 11 is at rest.

In a special embodiment the probe is moved by a micromechanic system essentially in a straight line in the occlusal plane (2 degrees of freedom). To adjust the focus the probe itself can be moved relative to a mirror or other deflection means or in direct scan. The mirror can be embodied in a tiltable manner, in order to be able to better detect marginal areas.

Through the new type of constellation in which the probe 11 during the actual measurement does not perform any straight-line displacements and in an initialization phase, depending on the embodiment and use, performs only small straight-line displacements, virtually any coupling means can be used. Therefore probes that emit longitudinal waves as well as those that emit transverse waves can be used.

Film resonators and composite resonators are particularly suitable for the use according to the invention, wherein film resonators are currently preferred.

The principle of the invention is described below based on several embodiments.

A first apparatus 10 according to the invention for data acquisition in the mouth of a patient is indicated diagrammatically in FIG. 1. The apparatus 10 comprises an ultrasound sensor 20 that is shown in section. Ultrasound deflection means 12 are provided that can be moved remotely as indicated by the dashed arrow 15. In order to couple the ultrasound sensor 20 acoustically to the tooth area 1, 2, 3 or residual tooth area to be scanned, a coupling body 21 with coupling means 21.1 or a damping means is provided. As indicated in FIG. 1, the coupling body 21 filled with the coupling means 21.1 or comprising the coupling means 21.1 or the damping means is located between the ultrasound deflection means 12 and the tooth area 1, 2, 3 or residual tooth area to be scanned. The coupling body 21 preferably comprises a thin, membrane-like casing or wall in order to retain the coupling means 21.1 contained therein. Gel-like or viscous means that are acoustically transparent for the ultrasonic waves in the desired range are particularly suitable as coupling means 21.1. Ultrasonic gel or agar agar are particularly preferred as coupling means. Since, depending on the embodiment of the coupling body 21, a part of the coupling means 21.1 can emerge in the mouth of the patient, a coupling means is preferred that is not harmful to health if swallowed.

The body 21, which contains or encloses the further coupling means 21.1, can preferably comprise a high-damping material in order to minimize artifacts by undesirable reflections.

The ultrasound deflection means 12 is embodied in a preferred embodiment such that a focusing of the ultrasonic wave (analogous to a parabolic reflector) is also generated through its form.

FIG. 1 shows three adjacent teeth 1, 2 and 3 (also known as a row of teeth), wherein the center tooth 2 (also called the residual tooth area) has been prepared, for example, in order for a crown to be added later. The gingiva is not shown.

Furthermore, means are provided (not shown in FIG. 1) in order to act on the ultrasound sensor 20 remotely with excitation signals, as indicated by the double arrow 14 in FIG. 1. These means also make it possible to move the ultrasound deflection means 12 (see arrow 15). Through the triggering of the ultrasound sensor 20 an ultrasonic wave is generated that is deflected and/or focused by the ultrasound deflection means 12 in the direction of the tooth area 1, 2, 3 or the residual tooth area. At least part of the ultrasonic wave is reflected there and fed back into the ultrasound sensor 20 via the ultrasound deflection means 12. The reverse ultrasonic wave is also referred to as an echo. In this case, the apparatus also functions in the pulse-echo method.

As shown in FIG. 1, the probe 11 together with the ultrasound deflection means 12 is located in a housing 13 or in a corresponding part of the housing. In the area between the ultrasound deflection means 12 and the tooth area 1, 2, 3 or residual tooth area to be scanned, a type of window is provided in the housing, which window is permeable at least for ultrasonic waves (i.e. acoustically transparent). This window cannot be seen in FIG. 1.

Through a suitable processing of the electrical excitation signals and the electrical signals that are emitted by the ultrasound sensor 20 upon receipt of the echo (called reflection signals), information can be obtained on the surface, for example, of the prepared tooth 2 in FIG. 1.

In the embodiment shown in FIG. 1, the housing 13, or the corresponding part of the housing, is at rest with respect to the tooth area 1, 2, 3 or the residual tooth area to be scanned. That means that neither the housing 13 nor the probe 11 makes a movement during the scanning (apart from the vibration movement, of course, which is generated by the electrical excitation signals). Depending on the size, shape and type of the area to be scanned, it is sufficient for the ultrasound deflection means 12, in FIG. 1 a small prismatic body to which a reflective coating is applied in a suitable manner, to be adjusted such that the area to be scanned is reached by an ultrasonic wave that is emitted by the probe 11 and deflected by the ultrasound deflection means 12. The resolution or focusing can be achieved, for example, by a straight-line displacement of the probe 11 inside the housing 13. This displacement is indicated in FIG. 1 by a double arrow below the probe 11. Through a displacement of this type, the distance from the ultrasonic deflection means 12 changes and thus the wave course is changed. Alternatively or additionally the distance between the probe 11 and the ultrasound deflection means 12 can also be changed in a different manner.

A focusing can also be achieved through a suitable form of the ultrasound deflection means 12, deviating from that of the prism.

In a currently preferred embodiment of the invention, the ultrasound deflection means 12 is tilted or rotated at least about one axis during the scanning procedure. In FIG. 1 an embodiment is indicated in which a rotation occurs about only one axis, namely the so-called mirror axis 12.1 (this mirror axis 12.1 stands perpendicular to the drawing plane).

This tilt movement is indicated by a double arrow D1. Through a controlled tilting movement of this type, the ultrasonic wave can also record at least in part the adjacent teeth 1 and 3.

In a currently preferred embodiment of the invention a tilting/rotation of the ultrasound deflection means about two axes is provided, in order to be able to scan the teeth from all sides.

Another embodiment of the invention is shown in FIG. 2. The same reference numbers are used for identical elements or elements acting in an identical manner. This also applies to all of the other embodiments of the invention. Since this embodiment is based on the embodiment shown in FIG. 1, or is a variant of the same, only the essential differences are described.

The probe 11 together with the ultrasound deflection means 12 is located in an elongated, rod-shaped housing 13 or housing part. The probe 11 can perform a straight-line displacement inside the housing 13, as indicated in FIG. 2 by a double arrow below the probe 11. A small reflector surface that can be rotated about a mirror axis 12.1 serves as ultrasound deflection means 12 (this mirror axis 12.1 stands perpendicular to the drawing plane). This tilt movement is indicated by a double arrow D1. A membrane 18 is provided as an ultrasound window on the exit or entry side of the housing 13. The head area of the housing 13 is surrounded by an optional body 17 that serves as a coupling element.

In the embodiment shown at least the outside of the housing 13 and the body 17 has a rotationally symmetrical form with respect to the longitudinal axis A-A. In addition to the adjustability of the resolution or focusing by a change of the distance between the probe 11 and the mirror 12, an area in the x direction can be swept by an ultrasonic wave on the one hand by pivoting the mirror 12 about the mirror axis 12.1. If another rotational axis or pivot axis is provided for the ultrasound deflection means 12, a deflection of the ultrasonic wave can also occur in the z direction. In this case, side views of the teeth 1, 2, 3 to be scanned are therefore also obtained.

In order to also obtain side views (which lie essentially in the x-y plane), for example, of the tooth 2 in an embodiment that has only a mirror axis 12.1, the ultrasound sensor 20 can be rotated about the longitudinal axis A-A.

Through a suitable combination of different rotational and swivel movements, larger areas can be recorded from different sides without having to shift or tilt the probe 11 during the scanning procedure, as is the case in the U.S. Pat. No. 6,050,821 cited above.

In a currently preferred embodiment of the invention the entire apparatus 10 or a part of the same can also be positioned in the x/z plane in a powered controlled manner, in order, for example, to be able to carry out an occlusal scan. In FIG. 5 this optional x/z movement is indicated by the double arrow D2.

An embodiment is particularly preferred which operates with the following 5 degrees of freedom, but is based on the principle previously described:
Straight-line motion of the probe 20 in x and z direction;
Straight-line adjustment of the probe 20 to vary the probe focus;
Rotational adjustment of the deflection means 12 (about 2 rotational axes).

Another embodiment of the invention is shown in FIG. 3 as a diagrammatic sectional representation. The section runs through the head area of the ultrasound sensor 20. In the sectional area the housing 13 has a circular cross section that is flattened on the lower side. An acoustically transparent membrane 18 is located in the area of the flattening. A mirror surface 12 serves as an ultrasound deflection means that can be rotated about a mirror axis 23 (this mirror axis lies in the drawing plane). The axis 23 runs parallel to the z direction, as indicated by reference number 12.1. In the example shown a rod 24, or means acting in the same manner, joins the lower right corner of the mirror surface 12, which rod tilts the mirror surface 12 about the axis 23 through a movement in the x direction. The rod 24 can run along the housing 13 on the inside from the head area up to the end so that it can thus be actuated outside the patient's mouth. This external actuation of the ultrasound deflection means 12 is indicated diagrammatically only by the dashed arrow 15 in FIGS. 1 and 2 previously discussed. However, in FIG. 3 a concrete realization example is shown for a corresponding actuation mechanism.

The mirror axis 23, as indicated, is suspended on the upper right and upper left in the housing 13. The exit side of the probe 11 is discernible behind the mirror surface 12 seen in the x direction.

The ultrasound sensor 20 can be rotated about the longitudinal center axis AA (indicated in FIG. 3 by a black dot in the center of symmetry), as indicated by the double arrow D3.

A so-called water coupling between the probe 11, the ultrasound deflection means 12 and the window, or the membrane 18 is particularly advantageous. A water coupling of this type can be achieved by filling the housing 13 with water (or a liquid of a similar type). Liquids are particularly preferred that are acoustically transparent in the desired wavelength range, i.e. that have a low acoustic resistance. A system with a coordinated selection of liquid and coupling means so that the boundary surface between the liquid and the coupling means is “acoustically transparent” is particularly preferred.

In a currently preferred embodiment of the invention water flows permanently through the head area labeled by 16 in FIG. 1 and FIG. 2. Preferably the water is supplied through a hose from outside the mouth. An embodiment is particularly preferred in which the supply occurs through the housing 13. Alternatively, water or another suitable liquid can also be simply present in the interior without flowing.

Details of another embodiment are shown in FIG. 4. This is a perspective, diagrammatic view of an ultrasound sensor 20 according to the invention in a patient's mouth. The same reference numbers are used for identical elements or elements with identical action. For the sake of clarity, only a part of the lower gingiva 7 and the upper gingiva 8 with respectively only three teeth 1, 2, 3 or respectively 4, 5 and 6 are shown. According to the invention a so-called support structure is used that is labeled by reference number 25 in FIG. 4. In the example shown this is a support structure 25 that is clamped and fixed between the upper jaw and lower jaw. The support structure 25 can be embodied in a manner similar to a bite insert that is clamped between the upper jaw and lower jaw of a patient in order to hold the x-ray film when taking x-ray images.

In a currently preferred embodiment of the invention a type of dental bite frame or tray is used as a support structure that is placed at or on the teeth in the patient's mouth before the data acquisition. This dental bite frame or tray is preferably essentially rigid, in the sense of non-elastic. Support structures are particularly preferred which absorb acoustic waves in order to thus be able to suppress interference echoes.

A corresponding example with a dental bite frame or tray as a support structure is shown in FIG. 5. The teeth of the lower jaw 26 are shown in this figure. The ultrasound sensor 20 is supported in a dental bite tray 27 that serves as a support structure. In FIG. 5 the dental bite tray 27 is shown in a transparent manner in order not to cover the teeth lying beneath. At the outer end of the ultrasound sensor 20 a thin hose 28 is discernible, through which the water can be fed into the ultrasound sensor 20. With embodiments that function without water rinsing, this hose 28 can be omitted. The probe 11 and the ultrasound deflection means 12 are controlled by signals that are guided through a multiconductor cable 29. Through the arrangement shown, the teeth 1, 2 and 3 can be easily be faultlessly detected without the ultrasound sensor 20 having to be moved relative to these teeth during the actual scanning operation.

Preferably a powered adjustability of the position of the ultrasound sensor 20 with respect to the dental bite tray 27 in the x-z plane (and thus also relative to the teeth to be scanned) is given, as indicated by the double arrow D2.

By closing the jaw, the support structure 25 (or 27) can be held still for the duration of the scanning operation, which is important for the scanning precision as well as the reproducibility of the measurements.

A fixed (defined) position of the ultrasound sensor 20 with respect to the tooth area 1, 2, 3 or residual tooth area is preset through the support structure 25 (or 27) and thus a relation to a buccal surface, labial surface, lingual surface and/or occlusal surface of a tooth is defined.

As indicated in FIG. 4, the ultrasound sensor 20 is mechanically connected to the support structure 25 (or to the dental bite tray 27 in FIG. 5). Analogous to FIG. 1, a small prismatic body 12 serves as ultrasound deflection means which can be pivoted about the axis 12.1. The pivot movement is effected by a rod 24, or a similar means. As indicated in FIG. 4, the rod 24 is guided to the end of the ultrasound sensor 20 and can be actuated there, for example, by means of an actuator, motor or a means acting in a similar manner. Instead of an electrically powered drive, for example, a hydraulic drive can also be used.

Moving the ultrasound deflection means 12 by means of hybrid kinematic means in order to sweep over the desired (tooth) area with an ultrasonic wave has proven particularly useful.

The apparatus 10 preferably comprises a housing 13 that is anatomically adapted to the conditions in a patient's mouth. An elongated, cylindrical housing 13 is particularly preferred. Closed housings are preferred, since they are easier to disinfect and can therefore be used several times.

In a further embodiment a thin film or a thin rubber 60, which is sterile, is drawn over the housing 13 before the housing is placed in the mouth. This film or this rubber 60 can serve as a membrane 18. A film or a rubber 60 that is elastic is preferred. A corresponding illustrated embodiment is shown in FIG. 9. The apparatus shown in FIG. 9 is a probe 20 that is filled with water or another liquid (i.e. does not need a hose connection) and receives and emits signals via the cable 29. Ultrasonic waves (US) are emitted through the rubber casing 60 as indicated diagrammatically in FIG. 9.

In another embodiment, an ultrasound source and a separate ultrasound receiver are used in the housing 13. This embodiment can be combined with all of the other cited embodiments.

In another embodiment, the ultrasound sensor 20 comprises a transceiver array probe with at least two transmitter elements and two receiver elements that are preferably arranged in a linear manner as an array.

In another embodiment, the ultrasound deflection means 12 are supported and moveable (for example, about 2 axes) such that they can be pivoted along a predetermined spatial curve. Special areas of a tooth or of several teeth can thus be scanned better, for example.

Micromechanical mirrors of silicon that can be moved by the application of electric control signals are particularly suitable as ultrasound deflection means 12.

The apparatus 20 can be designed such that a depiction of the recorded tooth or residual tooth area is carried out in black and white or in color.

Now that the fundamental features of the invention have been explained and various embodiments have been described, the corresponding method of data acquisition in the mouth of a patient with an ultrasound sensor 20 is described below. In this connection we refer to FIG. 6 that shows some of the essential process steps as a flow chart.

Before the ultrasound sensor 20 can be placed in the mouth of a patient, certain preparation steps are carried out, as indicated in FIG. 6 by reference number 31. Within the scope of this preparation, for example, a body 17 and/or coupling body 21 (for example a gel body) or a damping means, can be arranged in the patient's mouth or on the support structure 25. With an embodiment that uses a type of dental bite frame or dental bite tray 27 as a support structure, for example, a gel can be inserted as a coupling means into the dental bite frame or the tray 27. In FIG. 4 the coupling body filled with gel is labeled by reference number 21. The coupling body 21 and/or the gel fill a part of the tray 27.

Then in step 32 the ultrasound sensor 20 together with the support structure 25 (or tray 27) is placed into the patient's mouth and fixed there. The fixing can be carried out, for example, by biting down or closing the jaw. The apparatus 20, however, can also be temporarily attached to the patient's head with a headgear (as used by orthodontists). Subsequently, an optional calibration step or initialization step 39 can take place, for example. Within the scope of this step, for example, the resolution or the focusing can be adjusted by straight-line displacement of the probe 11 in the housing 13, or other adjustment movements can be carried out.

Now the ultrasound sensor 20 is acted on with electrical excitation signals (step 33), in order to emit an ultrasonic wave, and the ultrasound deflection means 12 is moved (step 34) in order to sweep over the area to be scanned with the emitted ultrasonic wave (this process is also referred to here as a scan movement). The movement of the ultrasound deflection means 12 can be carried out at the same time as the excitation of the probe 11, but this is not mandatory.

If only the deflection means 12 is moved, the wave front can be moved along the x axis or, in other words, this movement of the wave front is pivoted about an axis that runs parallel to the z axis. If other axes are present, a movement of the deflection means 12 about these axes and thus a corresponding deflection of the wave front can also be carried out.

Depending on the embodiment of the invention and the purpose, the housing 13 or a part of the housing can now be rotated (step 40) at the same time as step 34 and/or subsequently in order to also “expand” the scanning in a different direction. This can be carried out, for example, in order to thus also be able to detect buccal and labial tooth surfaces (better or more precisely). Instead of this mechanical rotation of the housing, this “expansion” can also be achieved with corresponding embodiment of the deflection means 12 by a suitable movement of the same. However, a movement in the x-z plane can also be made, as indicated in FIG. 5.

Depending on the movement of the ultrasound deflection means 12 and/or the housing 13, a scanning of the tooth area 1, 2, 3 or the residual tooth area can be achieved parallel to a row of teeth and/or perpendicular to the row of teeth.

The reflected ultrasonic waves are guided back at least in part to the housing 13 again and received there (step 35). The ultrasound sensor 20 provides corresponding electrical reflection signals (step 36) that are then processed in step 37 together with the excitation signals in order to thus render possible information on the surface, for example, of a tooth 2.

In the example shown, the apparatus 10 then provides a data set that can be transferred to a different system, or that can be further processed (step 38).

Steps following these steps are shown in FIG. 7.

The apparatus 10 according to the invention and the corresponding scanning strategy can be expanded such that then additional scanning planes are used when it is a matter of increasing the information density for the representation of edges and/or oblique surfaces and/or transitions and/or shadowed areas and/or undercuts and/or blind spots or other problem areas. In this case, the scanning strategy provides that in a first approach the relevant area is recorded in a rough scan (also called an overview scan). In the evaluation of the data set obtained, a corresponding software module determines whether edges, oblique surfaces, transitions, shadowing, undercuts, blind spots or other problem zones are present. This is labeled step 41 in FIG. 7. If this is the case, the scanning strategy is automatically adjusted accordingly (step 43) and additional images are made that can provide further data sets. This operation is summarized as step 44 in FIG. 7.

Within the scope of the additional images, depending on the type of problem zone the resolution can be increased, the position or the movement of the ultrasound deflection means 12 can be changed or the ultrasound sensor 20 can be rotated or positioned differently.

A scanning strategy has proven to be particularly useful in which the relevant area is detected with a rough scan in a first pass. Then a more precise pass follows with higher resolution. This more precise scan can, for example, cover only the prepared tooth 2, while all of the teeth 1, 2 and 3 are detected with the rough scan. Within the scope of the evaluation, the corresponding data sets are then assembled such that one overall image is produced in which the area of the tooth 1 is present with higher resolution. The assembly is carried out automatically (i.e. not manually) with the aid of 3D matching.

If the evaluation or processing of a data set shows that no problem areas are present, the data acquisition can be concluded, as indicated in FIG. 6 by step 42.

Depending on the embodiment of the apparatus 10, a water coupling can be carried out in an optional step 45. In FIG. 6 the water coupling is shown such that it begins before step 33 is carried out and is maintained at least as long as the reflected ultrasonic waves are received. In other words, the water coupling is carried out at least during part of the process steps. In the water coupling water is fed into the ultrasound sensor 20 in order to ensure a water coupling of the transmitter/receiver of the ultrasound sensor 20.

According to a further process, which is shown in FIG. 8, for example, two data sets are recorded by the dentist. A first data set is recorded in a first step 51 before the tooth or teeth are processed in the patient's mouth in a second step 52. This first data set as it were records the actual situation. Now, for example, the preparation of a tooth is carried out by means of grinding (step 52). After the preparation has been completed, an image can be recorded again with the apparatus according to the invention (step 53). Within the scope of this recording, a second data set is produced that comprises at least the prepared tooth.

One or both data sets can now be used in order to produce a crown or another dental prosthesis part. For example, the data can be sent to a milling center or laboratory, for example, after transmission by e-mail (step 54) so that the crown can be produced there (step 55). Based on the second data set, the shape of the contact area between the prepared tooth and the inside of the crown can be determined and produced. The first data set can be used, for example, when it is a matter of producing the external form of the crown. After the dental prosthesis part (e.g. in the form of a crown) has been produced, it is delivered to the dentist (step 56). The dentist then inserts the dental prosthesis into the patient (step 56). In this connection, typically a reworking is carried out, as indicated as step 58 in FIG. 8. Within the scope of the reworking, the dental prosthesis part can be given the final shape by grinding, polishing or other processing. For example, the color can also be changed.

However, the methods described can also be used for diagnosis. In this case, an area to be examined is recorded by means of the ultrasonic waves for diagnostic purposes and the result displayed or printed out. The attending physician can then evaluate the display or the printout in order to determine a suitable treatment. With the apparatus 10, for example, decayed areas of teeth or tartar can be detected based on the different reflective behavior. However, cracks, splits or the like can also be displayed.

Preferably an evaluation unit is used that is designed such that it can carry out a geometry determination of the tooth area or of the residual tooth area. An embodiment is particularly preferred in which a data set is processed by a special software in order to determine the geometry of the tooth area or of the residual tooth area. The evaluation unit can also or additionally determine a scatter plot that represents the surface of the tooth area or of the residual tooth area. Additionally or alternatively, based on the data set the evaluation unit can determine the topography of at least a part of the tooth area or of the residual tooth area.

However, the evaluation unit can also be designed such that it processes the data set in order to determine a 3D data set that can be used as a 3D surface model of the tooth area or of the residual tooth area.

In a further embodiment, in addition to the directly reflecting acoustic waves, scattered waves can also be received, processed and evaluated.

The invention can be easily used for detection in the front teeth area as well as in the rear molar area.

According to the invention, (artificial) cavities in a tooth can also be detected in order to produce a suitable inlay, for example.

In another embodiment the invention is used in connection with an apparatus according to European Patent 0 913 130 B1, wherein the apparatus described in the cited patent is used for scanning already (pre-) processed dental prosthesis parts (for example, in the laboratory) and the apparatus 10 according to the invention itself is used with the patient for data acquisition.

The invention can also be used particularly advantageously in connection with the method described in European patent application EP 1 062 916 in the production of implant-supported dental prosthesis.

With the invention one or more of the following elements can be processed, produced or the processing or production thereof can be supported:
removable or fixed dental prosthesis;
a crown structure;
a bridge structure;
an implant supraconstruction;
a dental brace;
a removable or fixed dental prosthesis, or
another orthodontic element.

The advantages of the invention are summarized below, wherein certain aspects are more important or are less marked, depending on the embodiment. The application of the invention is completely harmless for the patient as well as for the dentist and others involved. The method can be repeated as often as desired and is therefore also suitable for studies and the like. The method is non-invasive and absolutely painless. Compared to conventional methods, the treatment time is considerably reduced and error sources excluded as far as possible.

Expensive impression materials can be omitted through the use of the invention, which saves costs and is environmentally sound. The method is far less susceptible to faults than approaches hitherto known. Moreover the recording times are very short, which increases productivity.

Compared to previous approaches, the apparatus or the entire system is low-priced. This applies in particular when a single probe is used as transmitter/receiver. The application is thus more cost-efficient.

The invention thus has decisive advantages compared to the conventional impression technique as well as compared to CCD-based intraoral data acquisition.

The apparatus according to the invention can also be used particularly advantageously in connection with diagnosis, as already mentioned. Thus, for example, depending on the setting of the parameters (ultrasound frequency, etc.) the tooth surface can be scanned in order to detect cracks, splits, tartar or periodontosis.

One advantage of the apparatus according to the invention is that it does not require any means to determine the position of the ultrasound sensor in the patient's mouth or with respect to the teeth to be scanned. In other words, the apparatus is self-sufficient and does not need any auxiliary systems or the like.

Another advantage of the invention is that the essential mechanical/technical and electrical parts are accommodated in a housing 13 or in a part of the housing. This makes the entire system particularly sturdy and rugged. Moreover, the apparatus is particularly easy to clean and disinfect. Damage is also virtually impossible. The apparatus is much more inexpensive than some of the solutions previously known, since it needs only one probe instead of the usual probe arrays.

### LIST OF REFERENCE NUMBERS

Tooth area 1, 2, 3
Teeth 1, 2 and 3
Teeth 4, 5 and 6
Gingiva 7
Apparatus 10
Probe 11
Ultrasound deflection means 12
Rotational axis 12.1
Housing 13
Double arrow 14
Arrow 15
Head area 16
(Damping) body 17
Membrane 18
Ultrasound sensor 20
Coupling body 21
Axial direction 22
Mirror axis 23
Rod 24
Support structure 25
Lower jaw 26
Dental bite tray 27
Hose 28
Cable 29
Preparation step 31
Step 32
Step 33
Step 34
Step 35
Step 36
Step 37
Step 38
Calibration or initialization
step 39
Step 40
Step 41
Step 42
Step 43
Step 44
Optional step 45
Step 51
Step 52
Step 53
Step 54
Step 55
Step 56
Step 57
Step 58
60 film/casing

1. A method of acquiring data in the mouth of a patient by means of an ultrasound sensor wherein the ultrasound sensor comprises ultrasound deflection means and a support structure to hold the ultrasound sensor, the method comprising the following steps:
placing the support structure to hold the ultrasound sensor in the patient's mouth, wherein the ultrasound sensor is at rest during the data acquisition with respect to the tooth area or residual tooth area,
acting on the ultrasound sensor with excitation signals in order to generate an ultrasonic wave,
moving the ultrasound deflection means such that the ultrasonic wave sweeps over at least one tooth area or a residual tooth area,
recording waves reflected on the tooth area or residual tooth area by means of the ultrasound sensor or a separate ultrasound receiver and providing corresponding reflection signals,
providing the excitation signals and reflection signals for an evaluation unit, and
producing a data set using the evaluation unit from the excitation signals and reflection signals, wherein the data set is suitable for transfer to a processing system or an imaging system.

2. The method according to claim 1 wherein the ultrasound sensor is an intraoral scanner, the scanning movement of which is generated by the movement of the ultrasound deflection means.

3. The method according to claim 1 wherein the ultrasound sensor is an imaging ultrasound sensor.

4. The method according to claim 1 wherein the ultrasound sensor is an ultrasound sensor with separate transmitter and ultrasound receiver.

5. The method according to claim 1 wherein the ultrasound sensor is an ultrasound sensor with a transmitter that operates in the frequency range between 1 and 80 MHz.

6. The method according to claim 1 wherein the ultrasound sensor comprises a piezo ultrasound converter and a receiver, the resonance frequency of which lies in the range between 1 and 80 MHz.

7. The method according to claim 1 wherein the ultrasound sensor is designed such that through the movement of the ultrasound deflection means it renders possible a scanning of the tooth area or residual tooth area parallel to a row of teeth and perpendicular to the row of teeth.

8. The method according to claim 1 wherein the ultrasound deflection means comprise reflective means that are moved such that the ultrasonic wave is guided by the reflective means such that it sweeps over the tooth area or the residual tooth area.

9. The method according to claim 8 wherein the reflective means can be controlled and moved such that they can be pivoted along a predetermined spatial curve.

10. The method according to claim 1 wherein the ultrasound sensor comprises reflective means as ultrasound deflection means that are moved by means of hybrid kinematic means such that the ultrasonic wave is guided by the reflective means such that it sweeps over the tooth area or the residual tooth area.

11. The method according to claim 1 wherein
the evaluation unit carries out a geometry determination of the tooth area or the residual tooth area, and/or
the evaluation unit detects a scatter plot that represents the tooth area or the residual tooth area, and/or
the evaluation unit determines data that describe the topography at least of a part of the tooth area r the residual tooth area.

12. The method according to claim 1 wherein the evaluation unit processes the data set in order to determine a 3D data set that can be used as a 3D surface model of the tooth area or of the residual tooth area.

13. The method according to claim 1 wherein the evaluation unit processes the data set in order to be able to use it for the production or processing of
a crown structure, or
a bridge structure, or
an implant supraconstruction, or
a dental brace, or
a removable or fixed dental prosthesis, or
another orthodontic element.

14. The method according to claim 1 wherein the evaluation unit processes the data set in order to be able to use it for diagnosis.

15. The method according to claim 1 wherein a coupling body or a damping means, for example a gel body, is arranged between the ultrasound sensor and the tooth area or residual tooth area wherein this coupling body or this damping means is preferably held at or on the support structure.

16. The method according to claim 1 wherein the support structure is a type of dental bite frame or tray and this is placed at or on teeth in the patient's mouth before the data acquisition.

17. The method according to claim 1 wherein through the support structure a fixed position of the ultrasound sensor is preset with respect to the tooth area or residual tooth area in order to thus define a relation to a buccal surface or occlusal surface of a tooth.

18. The method according to claim 1 for the non-invasive three-dimensional recording of the tooth area or of the residual tooth area.

19. The method according to claim 1 wherein the data set is recorded with a lateral precision between 50 and 100 μm.

20. The method according to claim 1 wherein the ultrasound sensor is controlled such that then additional scanning planes are used when it is a matter of increasing the information density for the representation of edges or oblique surfaces or transitions or other problem zones.

21. The method according to claim 1 wherein at least during some of the steps water is supplied to the ultrasound sensor in order to provide a water coupling of the ultrasound sensor.

22. The method according to claim 1 wherein in a first phase an initial scan is carried out and then a second scan is carried out that has a higher resolution at least at one location.

23. The method according to claim 1 wherein an edge analysis is carried out and the process flow is then automatically adjusted to the current conditions.

24. The method according to claim 1 wherein the data set is transferred directly or indirectly to a milling system wherein the transfer is carried out via a network and the milling system is located in a milling center or dental laboratory.

25. An apparatus for acquiring data in the mouth of a patient, the apparatus comprising:
an ultrasound sensor and a support structure the ultrasound sensor being supported at rest by the support structure and having ultrasound deflection means that are moveable,
a coupling body or a damping means arranged between the ultrasound deflection means and a tooth area or residual tooth area to be scanned when the support structure together with the ultrasound sensor is placed in the patient's mouth, and
means for acting on the ultrasound sensor with excitation signals and for moving the ultrasound deflection means in order to thus generate an ultrasonic wave that sweeps over at least a part of the tooth area or residual tooth area.

26. The apparatus according to claim 25 wherein the ultrasound sensor comprises a probe that together with the ultrasound deflection means forms an assembly wherein the probe is positioned with respect to the ultrasound deflection means such that an ultrasonic wave generated by the probe by excitation with the excitation signals is guided from the probe in the direction of the ultrasound deflection means and from there through the coupling means or the damping means to the tooth area or residual tooth area.

27. The apparatus according to claim 25 wherein the probe and the ultrasound deflection means form an elongated assembly, preferably an essentially cylindrical assembly.

28. The apparatus according to claim 25, further comprising with
a drive unit for moving the ultrasound deflection means hydraulically or mechanically.

29. The apparatus according to claim 25 wherein the ultrasound sensor is an ultrasound sensor with separate transmitter and ultrasound receiver.

30. The apparatus according to claim 25 wherein the ultrasound sensor is an ultrasound sensor with a transmitter that operates in the frequency range between 1 and 80 MHz.

31. The apparatus according to claim 25 wherein the ultrasound sensor comprises a piezo ultrasound converter and an ultrasound receiver, the resonance frequency of which lies in the range between 1 and 80 MHz.

32. The apparatus according to claim 25 wherein the ultrasound sensor comprises a transceiver array probe with at least two transmitter elements and two receiver elements that are arranged in a linear manner as an array.

33. The apparatus according to claim 25 wherein the ultrasound deflection means comprise reflective means that can be moved from outside the mouth such that the ultrasonic wave is guided by the reflective means such that it sweeps over the tooth area or the residual tooth area.

34. The apparatus according to claim 33 wherein the reflective means can be controlled and moved such that they can be pivoted along a predetermined spatial curve.

35. The apparatus according to claim 25 wherein the ultrasound sensor comprises reflective means as ultrasound deflection means that can be moved from outside the mouth by means of hybrid, kinematic means such that the ultrasonic wave is guided by the reflective means such that it sweeps over the tooth area or the residual tooth area.

36. The apparatus according to claim 25 wherein the support structure is a type of dental bite frame or tray that can be placed at or on teeth in the patient's mouth before data acquisition.

37. The apparatus according to claim 25 wherein a water inlet is provided in order to render possible a water coupling of the ultrasound sensor.

38. The apparatus according to claim 25 wherein a gel body serves as a coupling body or damping means.

39. The apparatus according to claim 25 wherein the ultrasound sensor comprises an acoustically permeable membrane, film casing or rubber casing.

40. The apparatus according to claim 25 wherein it is integrated into or onto a dentist's chair.

41. A system with a dentist's chair and an apparatus according to claim 25 and with a data processing apparatus in order to provide data sets for transfer to a processing system or an imaging system.

42. Use of an apparatus according to claim 25 in connection with the production or processing of
a crown structure, or
a bridge structure, or
an implant supraconstruction, or
a dental brace, or
a removable or fixed dental prosthesis, or
another orthodontic element.

43. Use of an apparatus according to claim 25 for diagnosis.
